(19) United States
(12) Patent Application Publication
(10) Pub. No.: US 2007/0240709 A1
(43) Pub. Date: Oct. 18, 2007

(54) Multiple use nebulized oxygen delivery device and methods therefor

(75) Inventors: Leif Woolley, Archer, FL (US); Michael Golub, Boca Raton, FL (US)

Correspondence Address: CONNOLLY BOVE LODGE & HUTZ LLP, 1875 EYE STREET, N.W., SUITE 1100, WASHINGTON, DC, 20036, US

(73) Assignee: LMD Research and Development Inc., Boca Raton, FL (US)

(21) Appl. No.: 11/403,005

(22) Filed: Apr. 13, 2006

(51) Int. Cl. A61M 11/00

(52) U.S. Cl. 128/200.21

(57) ABSTRACT

The present disclosure relates to methods and devices for simultaneously delivering to a patient an inhalational gas comprising up to 100 fractional percent inspired oxygen and at least one nebulized medicine.

### FIELD OF THE INVENTION

The present disclosure relates to methods and devices for simultaneously delivering to a patient an inhalational gas comprising up to 100 fractional percent inspired oxygen and at least one nebulized medicine.

### BACKGROUND

The information provided below is not admitted to be prior art to the present invention, but is provided solely to assist the understanding of the reader.

The medical arts are familiar with devices and methods of delivering to a patient a stream of medical oxygen. Medical practitioners know that according to a patient's medical condition, it may be necessary to deliver a stream of inhalational oxygen at a flow rate high enough to ensure that the patient's blood is substantially 100% oxygenated.

The medical arts also know that according to a patient's medical condition, it may be necessary to deliver a stream of nebulized medicine to the patient. Conventionally, medicine is nebulized by the stream of inhalational oxygen. Convention inhalational devices permit only a single stream of gas to be administered to the patient. However, the flow rates appropriate to nebulizing medicines are often insufficient to maintain the patient's blood at the required 100% oxygenation. Medical personnel may be faced with the unfortunate choice of either delivering an appropriate level of oxygen, but an insufficient level of medication, or adequate medication, but an insufficient level of oxygen.

There exists an unmet need for a device that combines at least two streams of inhalational gas. There exists a need for a device that provides a first stream of inhalational oxygen and a second stream of nebulized medicine.

Other objects and advantages will become apparent from the following disclosure.

### SUMMARY OF INVENTION

An aspect of the invention provides a means of providing to a patient an inhalational gas comprising essentially 100% oxygen at a medically-appropriate flow-rate. A related aspect provides means of entraining a flow of a nebulized medicine within the flow of oxygen. An aspect provides means of providing a first stream of oxygen at a medically-appropriate flow-rate. A further aspect provides a means of providing a second stream of oxygen at a reduced flow-rate appropriate to nebulize a medicine. A related aspect provides means of combining the first and second streams into a single stream of inhalational gas.

An aspect of the invention provides a gas-stream entrainment device. An aspect provides the entrainment device is substantially a Y-tube having first and second Entrainment-inlet (E-inlet) ports and a single E-exit port.

An aspect provides that a single adaptor is suitable to interface with each of the patient masks likely to be used in a hospital. An aspect of the invention provides the E-exit port is configured such a single E-exit port is adapted to fit the inlet port of patient masks such as neonatal masks, infant masks, pediatric masks, juvenile masks, and adult masks.

An aspect provides that a single adaptor is suitable to interface with each of sources of inhalational gas likely to be used in a hospital. An aspect provides a primary oxygen inlet (POI) device. An aspect provides the POI device has a POI-exit port adapted to fit either inlet port of the entrainment device. An aspect of the invention provides that each POI-inlet port is adapted to fit the exit port of gas sources such as oxygen hoses and nebulized medicine hoses

An aspect provides means of combining into the primary oxygen stream a second stream of oxygen containing a nebulized medicine. An aspect of the invention provides a medicine delivery chamber (MDC) to interface between a source of nebulized medicine and the entrainment device. According to an aspect, the medicine delivery chamber has a MDC-exit port disposed to be received by any of the E-inlet ports of the entrainment device. According to an aspect, the medicine delivery chamber has a MDC-inlet port adapted to interface with substantially every type inhalational gas and/or nebulized medicine source likely to be found in a hospital. According to a further aspect, the MDC has a gas-tight valve disposed between the two MDC ports. The valve is adapted such that when the MDC is connected to a source of inhalational gas, the valve is in an open position to admit the passage of gas. When the MDC device is not connected to a gas source, the valve is disposed to be in a closed position that does not admit the passage of gas.

Still other aspects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BRIEF DESCRIPTION OF DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1A is a side elevation cross-section of a preferred embodiment highlighting the placement of the valve pin and valve pin pusher placed in the medicine delivery chamber of the inventive device;

FIG. 1B is a side elevation of the entrainment device;

FIG. 1C is a side elevation of the medicine delivery chamber of a preferred embodiment;

FIG. 1D is a side elevation cross-section of a preferred embodiment highlighting the placement of the valve spring placed in the medicine delivery chamber;

FIG. 1E is a side elevation of the primary oxygen inlet of a preferred embodiment;

FIG. 2A is a side elevation cross-section of a preferred embodiment of the medicine delivery chamber;

FIG. 2B is an exploded view of a preferred embodiment of the valve;

FIG. 3A is a side elevation of a preferred embodiment of the entrainment device indicating the Part Line for Section A-A;

FIG. 3B is a cross-section along line A-A of FIG. 3A;

FIG. 3C depicts a locking detent to engage a patient mask;

FIG. 3D is a bottom elevation of a preferred embodiment of the entrainment device;

FIG. 3E is a side elevation of a preferred embodiment of the entrainment device indicating the Part Line for Section B-B;

FIG. 3F is a cross-section along line B-B of FIG. 3E;

FIG. 3G is a cross-section of Detail 1 of FIG. 3E;

FIG. 3H is a side elevation of a preferred embodiment of the entrainment device;

FIG. 4A is a side elevation of a preferred embodiment of the primary oxygen inlet;

FIG. 4B is a cross-section of Detail 1 of FIG. 4D;

FIG. 4C is a side elevation of a preferred embodiment of the primary oxygen inlet indicating the Part Line for Section A-A;

FIG. 4D is a cross-section along line A-A of FIG. 4C;

FIG. 5A is a side elevation of a preferred embodiment of a valve pin;

FIG. 5B is a top-side elevation of a preferred embodiment of a valve pin;

FIG. 5C is a bottom elevation of a preferred embodiment of a valve pin;

FIG. 5D is a side elevation of a valve spring;

FIG. 6A is a side elevation (rotated 90°) of a preferred embodiment of a valve pin pusher;

FIG. 6B is a top elevation of a preferred embodiment of a valve pin pusher;

FIG. 6C is a perspective view of a preferred embodiment of a valve pin pusher;

FIG. 6D is a side elevation of a preferred embodiment of a valve pin pusher;

FIG. 7A is a top elevation of a preferred embodiment of the medicine delivery chamber;

FIG. 7B is a side section of the medicine delivery chamber indicating Detail 2; and

FIG. 7C is a cross-section through Detail 2 of FIG. 7B.

It is to be noted, however, that the appended drawings illustrate only typical embodiments of this invention and are therefore not to be considered limiting of its scope, for the invention may admit to other equally effective embodiments.

### DETAILED DESCRIPTION OF A PREFERRED EMBODIMENT

Reference is made to the figures to illustrate selected embodiments and preferred modes of carrying out the invention. It is to be understood that the invention is not hereby limited to those aspects depicted in the figures.

A patient under medical care may have need of both inhalational oxygen at high concentrations and a high flow rates and also of nebulized medicine. As is known to persons of skill in the arts, gas flow rates required to appropriately nebulize a medicine are often too low to provide medically-appropriate oxygenation to a patient. For example, depending on a patient's medical condition, oxygen flow rates of at least 15 liters per minute may be required. However, to nebulize various medications lower flow rates of about 6 to about 8 liters per minute may be required. Doctors are often faced with a dilemma of either providing appropriate oxygenation in the absence of medication, or of providing medication, but at a flow rate that provides insufficient oxygenation. The present invention provides means and methods of simultaneously providing appropriate levels of both oxygen and medication.

Additionally, both patient masks and inhalational gas supplies may have different sized fittings necessitating stocking a variety of adaptors. The present invention provides adaptor means so that a single device may interface with most or all of the masks and gas supplies that are likely to be found in a hospital.

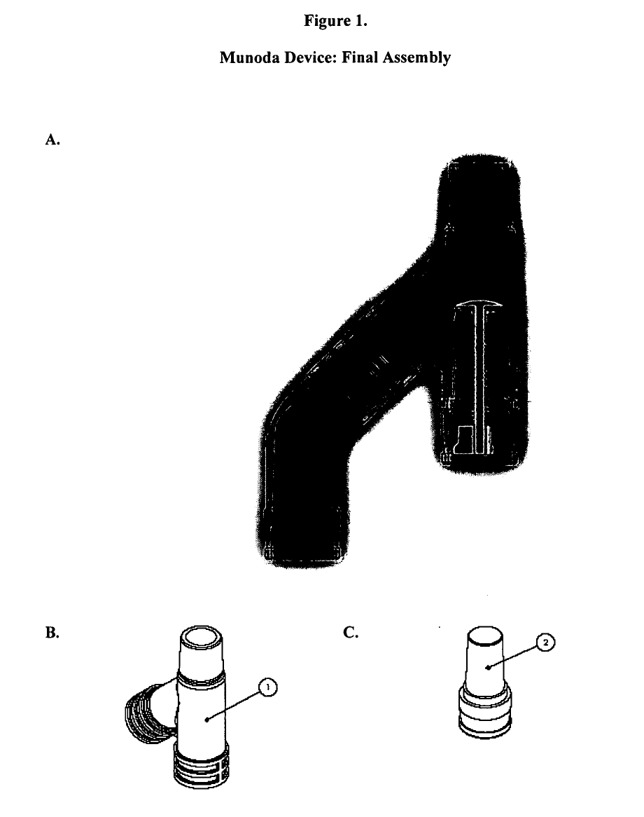

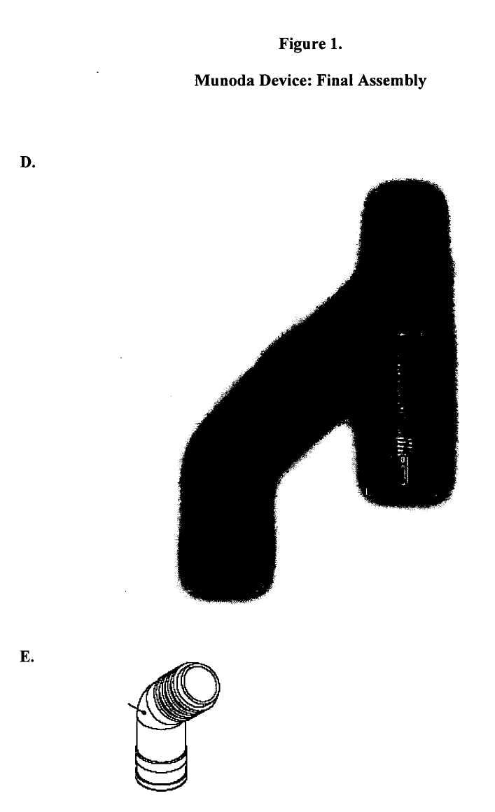

An aspect of the invention provides a manifold means for combining at least two streams of inhalational gas into a single stream. FIG. 1B depicts Y-tube 102 as a non-limiting embodiment of a manifold means. In this non-limiting embodiment, Y-tube 102 has two inlet ports 111, 113 and an outlet (exit) port 101. In a preferred embodiment, manifold means 102 has a main tube 108 having substantially co-axial inlet 113 and exit ports 101. At least one secondary tube 110 joins main tube 108. Preferably, the flow vector of the gas in the secondary tube(s) 110 meets the flow vector of the gas in main tube 108 at an acute angle. In a preferred embodiment, the flow vectors meet at about a 45° angle. The preferred embodiment as depicted in the Figures has a single secondary tube 110. However, embodiments having a plurality of secondary tubes 110 is encompassed by the invention. Manifold 102 may be termed an entrainment (E) means. Co-axial inlet 113 may be termed the E-inlet port, and the outlet may be termed the E-exit port 101. Each secondary tube has an inlet 111 offset from the axis of the main tube. The secondary inlet 111 may be termed the E-offset inlet port.

E-exit port 101 comprises a hollow cylinder, having an outer surface tapering outwardly to a decreasing diameter and adapted to be received by an inlet port of a patient mask. In an embodiment, the outer diameter of E-exit port 101 continuously tapers from its widest dimension at the proximal end (inside) to its narrowest dimension at the distal end (outside). In an embodiment, the outer diameter of E-exit port 101 tapers as a series of steps from its widest dimension at the proximal end to its narrowest dimension at the distal end. The dimensions and taper of E-exit port 101 are chosen such that the port readily engages patient masks including, but not limited to neonatal masks, infant masks, pediatric aerosol masks, adult simple masks, adult aerosol masks, venture masks, non-rebreather masks, partial rebreather masks, and face-tent masks.

Each E-inlet port 111, 113 comprises a hollow cylinder, having an inner surface tapering inwardly to a decreasing diameter and adapted to receive an inhalational gas stream means. Inhalational gas stream means are any of those known to persons of skill in the respiratory therapy arts. Such persons may include respiratory care practitioners, physicians, respiratory nurses, registered nurses, anesthesiologists, and emergency medical technicians. The inner surface of each inlet port 111, 113 may continuously taper, or may taper as a series of steps, from a widest dimension at the distal end (outside) to its narrowest dimension at the proximal end (inside). E-inlet port 111 is disposed to receive POI-Exit port 121. E-inlet port 113 is disposed to receive a medicine device chamber (MDC) or a secondary oxygen inhalational gas stream means.

FIG. 1E depicts a preferred embodiment in which a primary oxygen supply (not shown) is connected to manifold 102 through a primary oxygen inlet (POI) 106. Primary oxygen inlet 106 is substantially a hollow cylinder having a POI-inlet port 109 and a POI-exit port 121. In preferred embodiments, POI 106 has a bend or elbow 112 preferably, the bend is about 45°. POI-exit port 121 is adapted to form a gas-tight connection with manifold inlets 111 and 113. Preferably, POI-exit port 121 is connected to offset inlet 111.

In an embodiment, POI-exit port 121 comprises a hollow cylinder having an outer surface tapering outwardly to a decreasing diameter and adapted to be received by the Y-offset E-inlet port.

In an embodiment, POI-inlet port 109 comprises a hollow cylinder, having an inner surface tapering inwardly to a decreasing diameter and adapted to receive an inhalational gas stream means. The inner surface of each POI-inlet port 109 may continuously taper or taper as a series of steps.

An aspect of the invention provided a means of nebulizing a medicine with oxygen (or other inhalational gas as is medically appropriate) at a lower flow rate more appropriate to nebulization. A further aspect of the invention provides a means of combining a stream of nebulized medicine with a stream of medical oxygen. An aspect of the invention provides a medicine delivery chamber (MDC) connected to the manifold means.

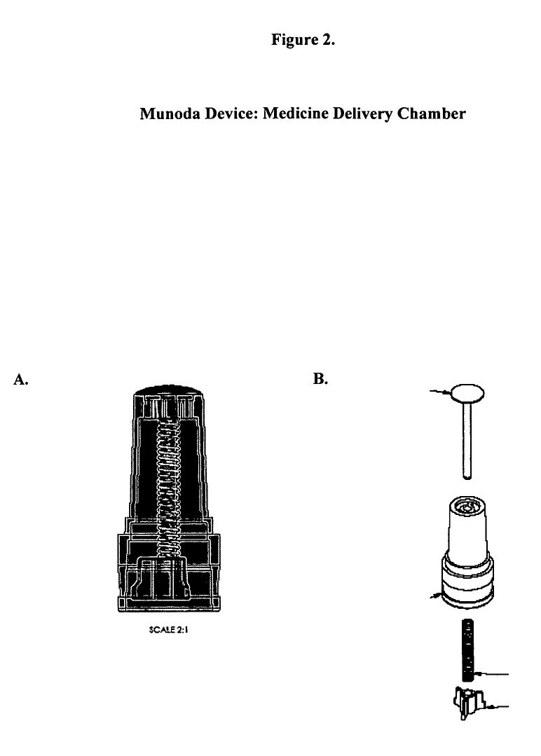

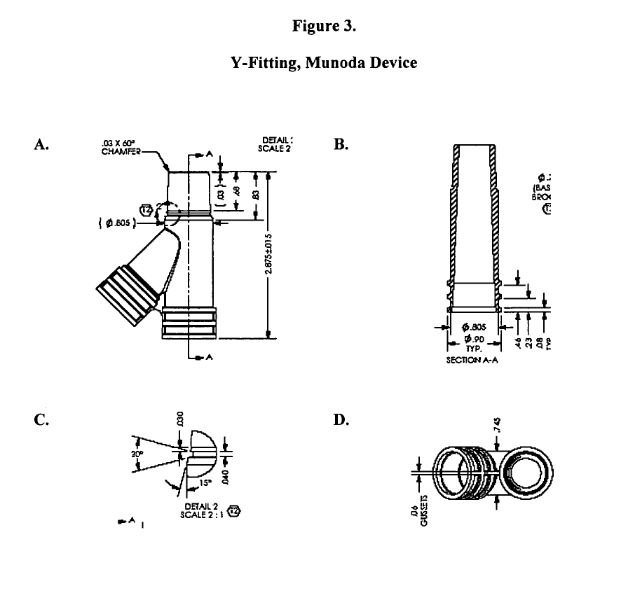

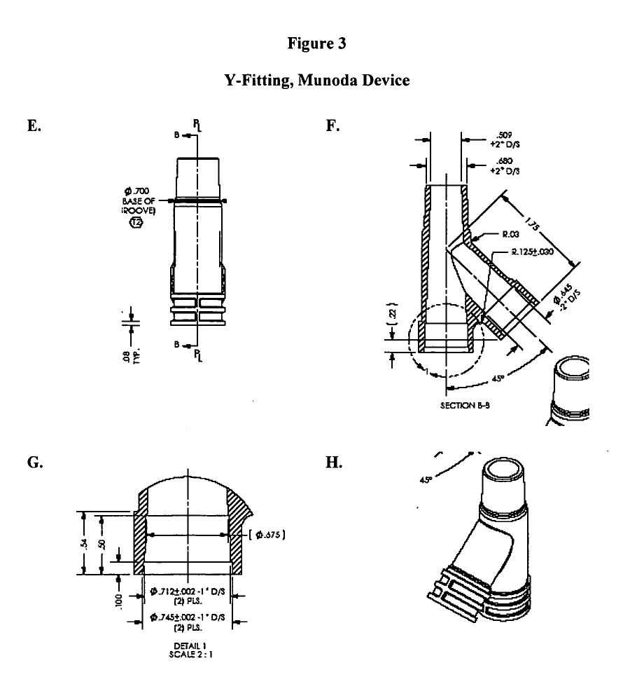

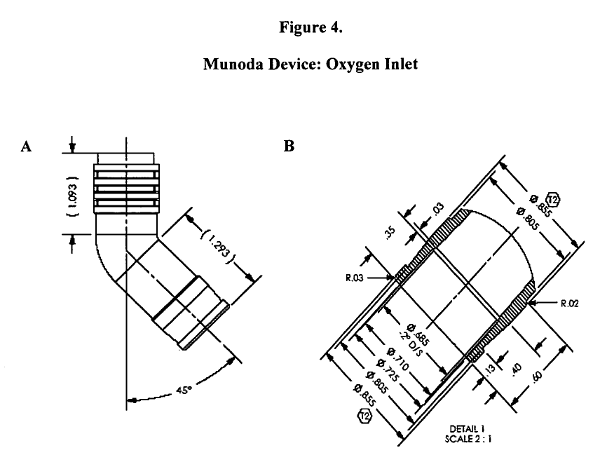

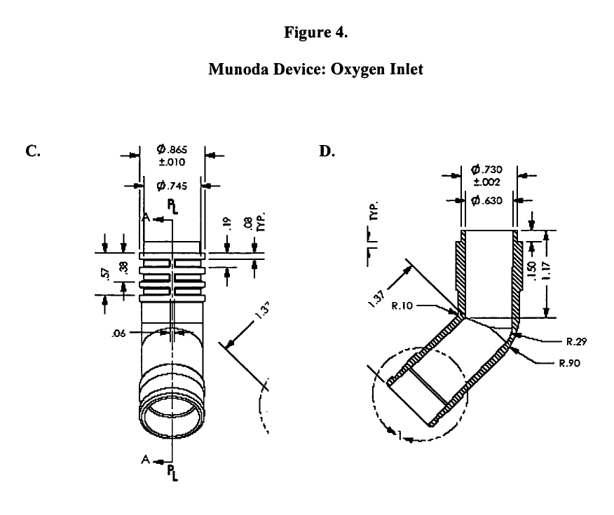

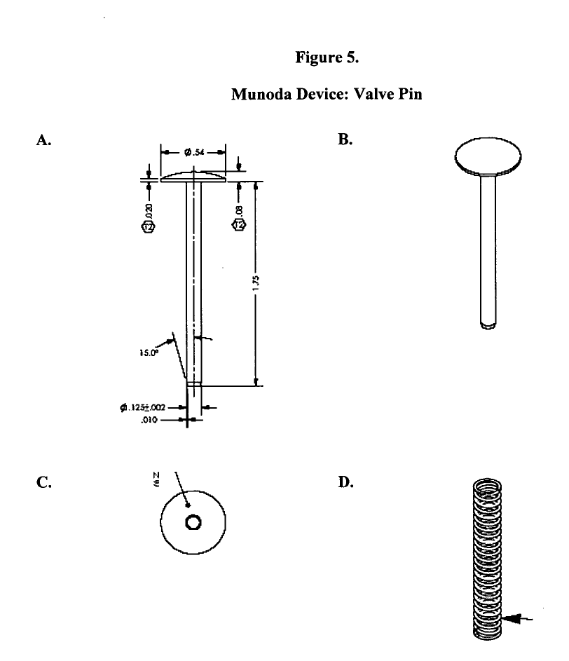

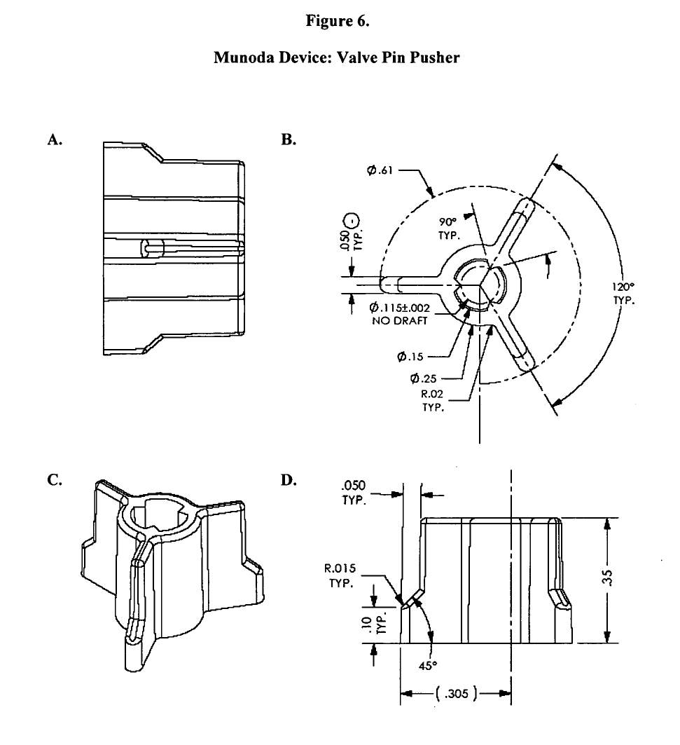

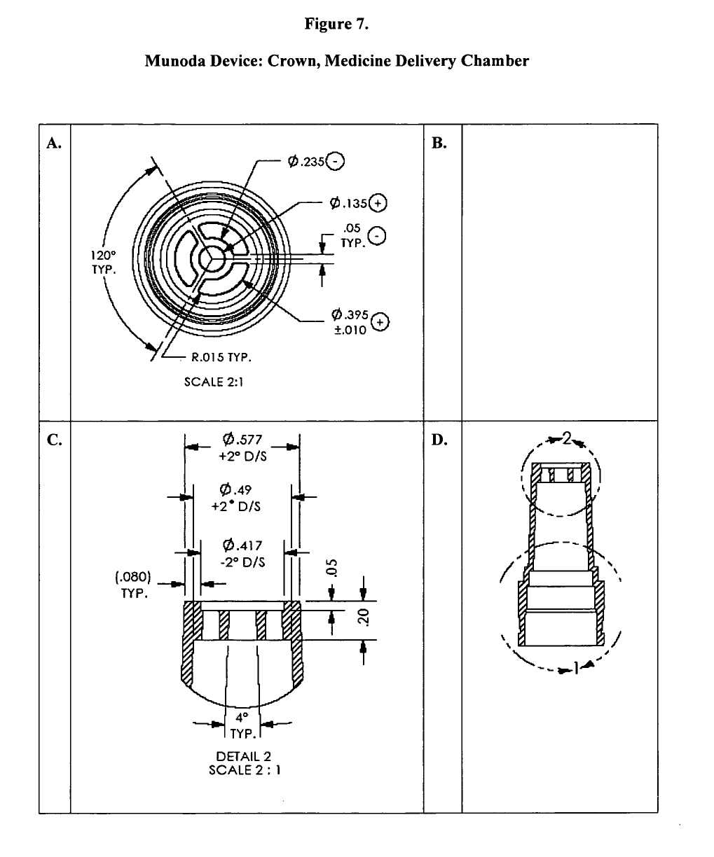

FIG. 2A is a side elevation cross-section of a preferred embodiment of medicine delivery chamber (MDC) 200. FIG. 2B is an exploded view of a preferred embodiment of valve means 202 contained within MDC 200. In a preferred embodiment, MDC 200 is substantially a hollow tube having MDC-inlet port 217 and MDC-exit port 215. Mounted internal to MDC 200, proximal to MDC-exit port 215 is crown 231. FIG. 7 highlights Crown 731 detail.

FIG. 7A shows crown 731 in top elevation as being substantially disk-shaped and having a plurality of passages defined therethrough. An axial passage 741 is disposed to admit valve pin 225. At least one passage 743 is disposed to admit an inhalational gas upon opening of valve 223. FIG. 7B shows a side elevation cross-section of crown 731. FIG. 7C is a side elevation cross-section of MDC 200 indicating crown 731 as Detail 2.

The present invention provides a method to treating a patient with high flow rates of inhalational oxygen together with a flow of nebulized medicine. In the inventive method a supply of medical oxygen is attached to a Primary Oxygen Inlet (POI). The supply may be directly from a tank or may be via intermediary devices. The POI directs the flow of oxygen to a first inlet port of an entrainment means. The flow of oxygen is a first stream of inhalational gas. The entrainment means may be a manifold.

A medicine delivery chamber (MDC) is attached to a second inlet port of the entrainment means. A second stream of inhalational gas may be attached to an MDC inlet port. The MDC is fitted with a valve means such that, in the absence of a second stream of inhalational gas, the valve is closed thus prohibiting the first stream (oxygen) from exiting away from the patient. A stream of nebulized medicine (second inhalational gas stream) may be coupled to the MDC inlet port. The second stream may be provided directly from a nebulizer. Alternatively, the second stream may be provided via one or more intermediary devices. The second stream may comprise a medicine nebulized in a stream of medical oxygen. The flow rate of the second stream may be less than the flow rate of the first stream.

The first and second streams may be entrained by the manifold entrainment means. The first stream of inhalational gas and/or the entrained first and second streams are directed through the manifold exit port to the patient. As may be medically indicated, one or more devices may be disposed between the manifold exit port and the patient.

### Incorporation by Reference

Throughout this application, various references including publications, patents, and pre-grant patent application publications are referred to. Disclosures of these publications in their entireties are hereby incorporated by reference into this application to more fully describe the state of the art to which this invention pertains. It is specifically not admitted that any such reference constitutes prior art against the present application or against any claims thereof. All publications, patents, and pre-grant patent application publications cited in this specification are herein incorporated by reference, and for any and all purposes, as if each individual publication or patent application were specifically and individually indicated to be incorporated by reference. In the case of inconsistencies the present disclosure will prevail.

### CD-ROM Appendix

Mechanical drawings, and other information, representing a preferred embodiment are submitted as an appendix on two CD-ROM disks. A first CD is labeled “CD 1 of 2: MUNODA”; a second CD is labeled “CD 2 of 2: Proto_{—}01.” Each CD is associated with Attorney Docket number 22347-00001-US and Representative Customer. Number 30678. Each CD and the entire contents thereon forms a part of the specification of the present application and is specifically incorporated by reference for all purposes.

1. A device comprising:
means for providing at least two inhalation gas streams;
means for providing at least a first stream of inhalational oxygen; and
means for entraining at least a first stream of a nebulized medicine.

2. A device according to claim 1, wherein:
the means for providing at least two inhalational gas streams comprises an entrainment (E) Y-tube further comprising a first E-inlet port co-axial with an E-exit port and a second E-inlet port Y-offset from the E-exit port, wherein
the E-exit port comprises a hollow cylinder, having an outer surface tapering outwardly to a decreasing diameter and adapted to be received by an inlet port of a patient mask, and wherein
each E-inlet port comprises a hollow cylinder, having an inner surface tapering inwardly to a decreasing diameter and adapted to receive an inhalational gas stream means;
wherein the means for providing at least a first stream of inhalational oxygen comprises a primary oxygen inlet (POI) further comprising:
a POI-exit port comprising a hollow cylinder having an outer surface tapering outwardly to a decreasing diameter and adapted to be received by the Y-offset E-inlet port, and
a POI-inlet port comprising a hollow cylinder, having an inner surface tapering inwardly to a decreasing diameter and adapted to receive an inhalational gas stream means; and
wherein the means for entraining at least a first stream of a nebulized medicine comprises a medicine delivery chamber (MDC) further comprising:
an MDC-exit port adapted to be received by the inline E-inlet port,
an MDC-inlet port comprising a hollow cylinder, having an inner surface tapering inwardly to a decreasing diameter and adapted to receive an inhalational gas stream means, and
a valve means disposed between the MDC-inlet and the MDC-exit ports.

3. The device of claim 2, wherein said patient mask is selected from the group consisting of neonatal masks, infant masks, pediatric aerosol masks, adult simple masks, adult aerosol masks, venture masks, non-rebreather masks, partial rebreather masks, and face-tent masks.

4. A method of simultaneously delivering to a patient an inhalational gas comprising up to 100 fractional percent inspired oxygen and at least one nebulized medicine, the method comprising:
entraining at least a first stream of inhalational gas comprising about 100% oxygen; and
entraining at least a second stream of inhalational gas comprising a nebulized medicine.

5. The method of simultaneously delivering to a patient an inhalational gas, according to claim 4, wherein the medicine is nebulized by a stream of gas comprising about 100% oxygen.

6. A method of providing a patient a high minute-ventilation and simultaneously a nebulized medicine comprising:
providing a means for entraining at least two inhalational gas streams, said means comprising an entrainment (E) Y-tube further comprising a first E-inlet port co-axial with an E-exit port and a second E-inlet port Y-offset from the E-exit port, wherein
the E-exit port comprises a hollow cylinder, having an outer surface tapering outwardly to a decreasing diameter and adapted to be received by an inlet port of a patient mask, and wherein
each E-inlet port comprises a hollow cylinder, having an inner surface tapering inwardly to a decreasing diameter and adapted to receive an inhalational gas stream means; wherein
providing a means for entraining at least a first stream of inhalational oxygen, said means comprising a primary oxygen inlet (POI) further comprising:
a POI-exit port comprising a hollow cylinder having an outer surface tapering outwardly to a decreasing diameter and adapted to be received by the Y-offset E-inlet port, and
a POI-inlet port comprising a hollow cylinder, having an inner surface tapering inwardly to a decreasing diameter and adapted to receive an inhalational gas stream means; and wherein
providing a means for entraining at least a first stream of a nebulized medicine comprises a medicine delivery chamber (MDC) further comprising:
an MDC-exit port adapted to be received by the inline E-inlet port,
an MDC-inlet port comprising a hollow cylinder, having an inner surface tapering inwardly to a decreasing diameter and adapted to receive an inhalational gas stream means, and
a valve means disposed between the MDC-inlet and the MDC-exit ports;
providing a first stream of inhalational oxygen in communication with said PIO; and
providing a stream of nebulized medicine in communication with said MDC-inlet port.

7. The device of claim 1 further comprising a valve means disposed between the means for providing at least a first stream of inhalational oxygen and the means for entraining at least a first stream of a nebulized medicine.

8. The device of claim 1 further comprising a chamber for combining the at least a first stream of inhalational oxygen and the at least a first stream of nebulized medicine.

9. A method of using the device of claim 8, wherein the at least a first stream of inhalational oxygen has a flow rate and the at least a first stream of a nebulized medicine has a lower flow rate.

10. The method of claim 4 wherein the at least a first stream of inhalational gas comprising about 100% oxygen has a flow rate and the at least a second stream has a lower flow rate.

11. The method of providing a patient a high minute-ventilation and simultaneously a nebulized medicine, according to claim 6, wherein the medicine is nebulized by a stream of gas comprising about 100% oxygen.
